(19) United States
(12) Patent Application Publication
(10) Pub. No.: US 2025/0217983 A1
(43) Pub. Date: Jul. 3, 2025

(54) Method for the analysis of radiographic images, and in particular lateral-lateral teleradiographic images of the skull, and relative analysis system

(71) Applicant: CEFLA S.C., Imola (Bologna) (IT)

(72) Inventors: Giuseppe Cota, Rovigo (RO) (IT); Gaetano Scaramozzino, Rovigo (RO) (IT); Giorgio Oliva, Rovigo (RO) (IT)

(21) Appl. No.: 18/851,039

(22) PCT Filed: Apr. 6, 2023
(86) PCT No.: PCT/IT2023/050100

§ 371 Date: Sep. 25, 2024

(30) Foreign Application Priority Data
Apr. 7, 2022 (IT) 102022000006905

(51) Int. Cl. G06T 7/00; A61B 6/00; A61B 6/46; A61B 6/50; G06T 3/40; G06T 3/60; G06T 5/92; G06T 7/11; G06T 7/60; G06T 7/73; G16H 50/20

(52) U.S. Cl. CPC G06T 7/0014; A61B 6/461; A61B 6/501; A61B 6/5205; A61B 6/5211; G06T 3/40; G06T 3/60; G06T 5/92; G06T 7/11; G06T 7/60; G06T 7/74; G16H 50/20; G06T 2207/10116; G06T 2207/20021; G06T 2207/20081; G06T 2207/20212; G06T 2207/30008; G06T 2207/30036

(57) ABSTRACT

A computer-implemented method for the geometric analysis of digital radiographic images, in particular lateral-lateral teleradiographs of the skull, uses a radiographic system that includes a display unit and processing system connected to the display unit. The radiographic system is configured for analyzing digital radiographic images.

The present invention relates to a method for the analysis di radiographic images, and in particular lateral-lateral teleradiographic images of the skull, and relative analysis system.

### FIELD OF THE INVENTION

In greater detail, the invention relates to a computer-implemented method for the analysis of lateral-lateral teleradiographs of the skull in order to detect anatomical points of interest and regions of interest. In fact, the invention relates to a method that, by means of techniques based on computer vision and artificial intelligence, enables an accurate analysis of radiographs in order to detect the position of anatomical points of interest which may be used to perform, by way of non-limiting example, cephalometric analyses in orthodontics.

The description below will be focused, as said, on the analysis of orthodontic images, but it is clearly evident that the same must not be considered limited to this specific use.

### PRIOR ART

As is well known, one of the most frequent orthodontic treatments is related to the treatment of malocclusions, which consist in a lack of stability of occlusal contacts, and in the lack of correct “functional guides” during masticatory dynamics. Malocclusions are also the cause of a highly unaesthetic appearance.

The diagnostic process requires the use of radiographic imaging, and in particular the execution of a lateral-lateral teleradiograph followed by a cephalometric analysis thereof.

Cephalometric analyses take on a fundamental importance also in the diagnosis and planning of orthodontic treatments or orthognathic surgical treatments (with the involvement, therefore, of other medical specialists such as a maxillofacial surgeon).

The first step of the analysis consists in the detection of anatomical points of interest in order to be able to define a cephalometric tracing and perform calculations of the angles and distances of the planes passing through the aforesaid anatomical points.

As is well known, in the medical-dental field, the identification of anatomical points of interest on a lateral-lateral teleradiograph of the skull is, in most cases, presently made by a doctor without any computerised support, except only the simple display of images and storage of information entered manually.

Once the aforesaid anatomical points of interest have been identified, on the market there exist various software systems, i.e. computer implemented programs, which make it possible to define a cephalometric tracing and automatically carry out a cephalometric analysis.

However, the detection of anatomical points on a radiograph is a highly time-consuming activity and is influenced by the level of experience and competence of the doctor who analyses the data, as well as his or her level of concentration and fatigue at the time of actually performing the analysis.

Furthermore, inexperience with particular anatomical sections and inattention could lead one to perform incomplete or incorrect diagnoses, or to prescribe wrong treatments.

It appears evident that the solutions and practices according to the prior art are potentially costly, because they can also create temporary or permanent damage to the patient's detriment.

### AIM OF THE INVENTION

In the light of the above, therefore, an aim of the present invention is to propose a system and a method for the analysis of lateral-lateral teleradiographs of the skull which overcome the limits of those of the prior art.

Another aim of the present invention is to propose a support system for doctors-radiologists, and dentists in particular, which enables the location and detection of anatomical points useful for cephalometric analysis.

A further aim of the present invention is to reduce as much as possible the risk of the dentist providing inaccurate or mistaken diagnoses, therapies and treatments.

### OBJECT OF THE INVENTION

Therefore, a specific object of the present invention is a computer-implemented method for the geometric analysis of digital radiographic images, in particular lateral-lateral teleradiographs of the skull, by means of a radiographic system, wherein said radiographic system comprises a display unit, and processing means connected to said display unit, said method comprising the steps of: performing, by means of said processing means, a learning step comprising the following sub-steps: receiving a plurality of digital learning radiographic images, each accompanied by annotations, wherein an annotation comprises a label identifying an anatomical point of interest of each learning radiographic image, and the geometric coordinates of the anatomical point of interest in the plane of the learning radiographic image; executing, by means of said processing means, for each learning radiographic image, a procedure for learning a general model for detecting one or more points of interest from a learning radiographic image, performing a refinement model learning procedure, comprising the sub-steps of: cutting the radiographic image into a plurality of image cutouts, each comprising a respective group of anatomical points of interest; and training a refinement model for each image cutout; and carrying out an inference step by means of said processing means on a digital analysis radiographic image, comprising the following sub-steps: performing on said analysis radiographic image an inference step based on said general model learned in said general model learning procedure, so as to obtain the geometric coordinates of a plurality of anatomical points of interest; cutting the analysis radiographic image into a plurality of image cutouts, in a similar way to said image cutting out step, wherein each image cutout comprises a respective group of anatomical points of interest; and performing on each cutout of the analysis radiographic image an inference through said refinement model obtained in said training step of said refinement model learning procedure; and combining the anatomical points of interest of each image cutout so as to obtain the final geometric coordinates of the points relative to the original analysis radiographic image; and displaying said final geometric coordinates of the points relative to the original analysis radiographic image by means of said display unit.

Again according to the invention, said learning step can comprise the sub-step of carrying out, by means of said processing means, for each learning radiographic image, a procedure for learning a radiograph cutout model for cutting out the part of the lateral-lateral teleradiograph of the skull that is relevant for the cephalometric analysis.

Likewise according to the invention, said step of carrying out said inference step can comprise the sub-step of performing, on said analysis radiographic image, an inference step based on said radiograph cutout model learned in said radiograph cutout model learning procedure, so as to obtain a cutout of the part of the lateral-lateral teleradiograph of the skull that is relevant for the cephalometric analysis.

Advantageously according to the invention, said method can comprise a step of performing on said analysis radiographic image an inference step based on said radiograph cutout model, which is carried out before said step of performing on said analysis radiographic image an inference step based on said general model learned in said general model learning procedure.

Furthermore, according to the invention, said general model learning procedure can comprise a first data augmentation step comprising the following sub-steps: random rotation of the radiographic image by a predefined range of angles with predefined probability; random horizontal flip, wherein the annotated acquired radiographic images are randomly flipped horizontally with a predefined probability; random contrast adjustment, wherein the image contrast is adjusted based on a predefined random factor; random brightness adjustment, wherein the brightness of images is adjusted based on a predefined random factor; random resizing and cutting out, wherein the radiographic image is resized with a random scale factor and cut out.

Again according to the invention, said general model learning procedure can comprise, before said general model learning step, a resizing sub-step.

Likewise according to the invention, said refinement model learning procedure can comprise the sub-steps of: performing a second data augmentation step; and executing said general model as obtained from said general model learning sub-step.

Advantageously, according to the invention, said second data augmentation step of said refinement model learning procedure can comprise the following sub-steps: random rotation, wherein each radiographic image and the relative annotations are rotated by a predefined range of angles and/or with a predefined probability, thereby generating a plurality of rotated images; horizontal flip of the radiographic images randomly annotated with a predefined probability; adjusting the contrast of said radiographic images based on a predefined random factor; and adjusting the contrast of said radiographic images based on a predefined random factor.

Furthermore, according to the invention, said step of training a refinement model for each image cutout can comprise the following sub-steps: resizing each cutout of said radiographic image; and performing a feature engineering and refinement model learning procedure; and/or performing a procedure for learning a dimensionality reduction model; and carrying out the refinement model learning.

Preferably, according to the invention, said step of performing a feature engineering and refinement model learning procedure can be based on computer vision algorithms, such as Haar or HOG, or on deep learning approaches, such as CNN or autoencoders.

Again according to the invention, said step of performing a dimensionality reduction model learning procedure can comprise Principal Component Analysis—PCA or Partial Least Squares regression—PLS.

Likewise according to the invention, said step of performing a feature engineering and refinement model learning procedure can comprise the following structure: a feature engineering model or procedure; and a set of regression models with the two-level stacking technique, comprising a first level, comprising one or more models; and a second level comprising the metamodel; and wherein at the output of said refinement model the coordinates of the group of anatomical points or points of interest of each cutout of said radiographic image are obtained.

Furthermore, according to the invention, said one or more models of said set of regression models can comprise at least one of the following models: support vector machine; and/or decision trees; random forest; and/or extra tree; and/or gradient boosting.

Advantageously according to the invention, said step of pre-processing said analysis radiographic image can comprise the following sub-steps: performing an adaptive equalization of a contrast-limited histogram, wherein the image is modified in contrast; and resizing the analysis radiographic image.

Preferably, according to the invention, said combining step of said inference step can comprise the steps of: aggregating and repositioning the anatomical points of interest, wherein the annotations returned by the refinement models are aggregated together with those of the original model, in such a way that the geometric coordinates of the anatomical points detected are relative to the original analysis radiographic image; reporting the missing anatomical points of interest, wherein it is reported whether there are points that have not been detected; carrying out a cephalometric tracing, wherein, based on the detected points, the tracing lines are defined; performing a cephalometric analysis, wherein, based on the detected points, one or more cephalometric analyses among those known in the scientific literature are performed.

A further object of the present invention is a system for analysing digital radiographic images, comprising a display unit, such as a monitor, and the like, and processing means, connected to said display unit, configured to carry out the analysis method as defined above.

An object of the present invention is also a computer program comprising instructions which, when the program is executed by a computer, cause the computer to execute the steps of the method as defined above.

Finally, an object of the present invention is a computer readable storage medium comprising instructions which, when executed by a computer, cause the computer to execute the steps of the method as defined above.

### BRIEF DESCRIPTION OF THE FIGURES

The present invention will now be described by way of non-limiting illustration according to the preferred embodiments thereof, with particular reference to the figures in the appended drawings, wherein:

FIG. 1 shows the steps of a method for the analysis of lateral-lateral teleradiographs of the skull according to the present invention when it operates in the learning (training) mode;

FIG. 2 shows a structure of a refinement model (with the feature engineering and dimensionality reduction step) of the method according to the present invention;

FIG. 3 shows the operating steps of the system according to the present invention in the inference operating mode;

FIG. 4 shows the sub-steps of the pre-processing step in FIG. 3;

FIG. 5 shows the sub-steps of the combining step in FIG. 3;

FIG. 6 shows an example embodiment of the graphic interface of the system for displaying the anatomical points detected;

FIG. 7 shows an example embodiment of the graphic interface of the system for displaying the cephalometric tracing constructed from the anatomical points detected;

FIG. 8 shows an example embodiment of the graphic interface of the system for displaying the cephalometric analysis constructed from the anatomical points detected; and

FIG. 9 shows a block diagram of a system for the analysis of radiographic images, and in particular lateral-lateral teleradiographic images of the skull, according to the present invention.

### DETAILED DESCRIPTION

In the various figures, similar parts will be indicated with the same numerical references.

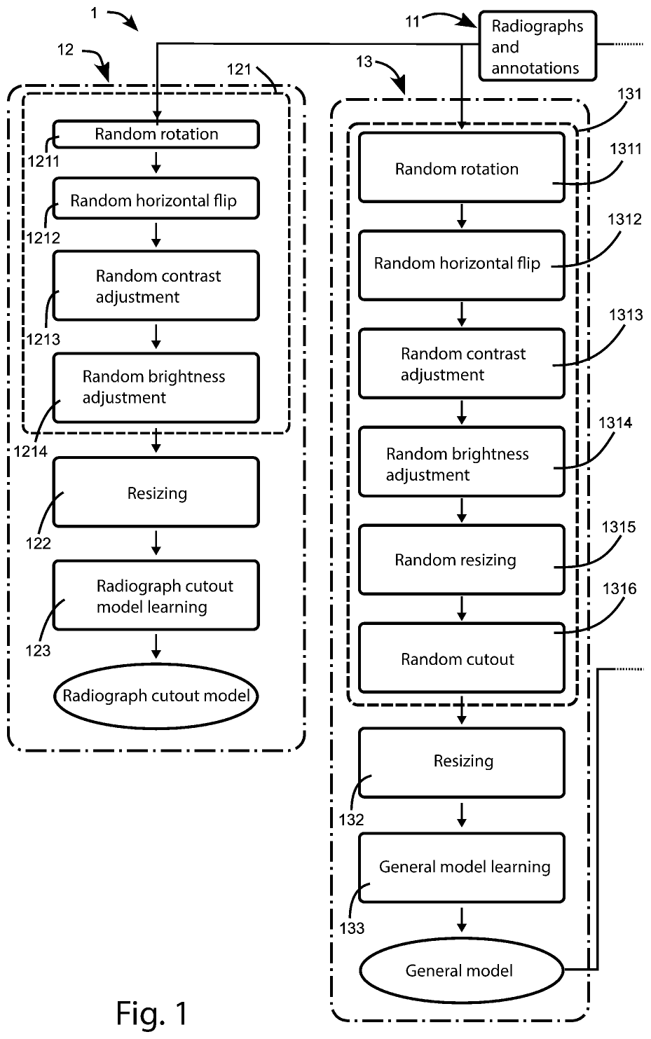

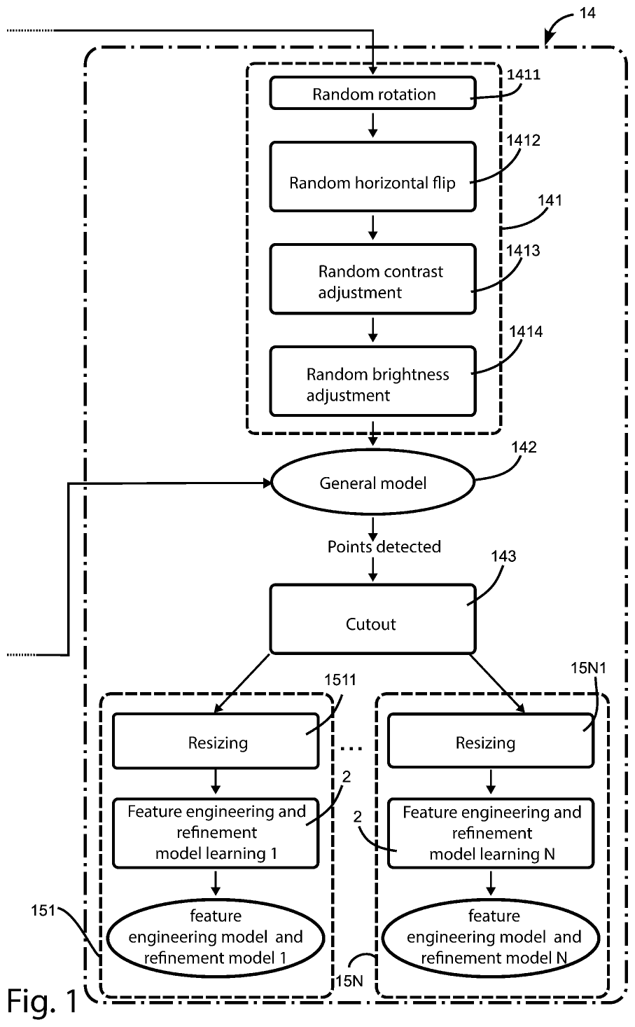

In general terms it is possible to distinguish, in the radiographic analysis method according to the present invention, two distinct modes or operating steps in which the system for the analysis of lateral-lateral teleradiographs of the skull works. In particular, also making reference to FIGS. 1-5, these operating modes are:
learning, in which the system learns, based on radiographic images and learning annotations, the operating modes for processing radiographs; and
inference, wherein the system receives radiographs for analysis and carries out the processing necessary in order to detect the anatomical points of interest, as will be better defined below.

In general, when the analysis method is in the learning mode, machine learning models are generated, which provide a set of radiographic images accompanied by annotations as input to the learning algorithms (better specified below).

For the sake of clarity in what follows, an annotation related to an element present in an image consists of two main components:
a label identifying the anatomical point of interest; and
the geometric coordinates of the anatomical point of interest in the plane of the image, i.e. of the radiograph.

Again in general terms, once the learning operating step has ended, the models thus trained are used, as mentioned earlier, in the inference operating step, i.e. in the actual utilisation of the analysis system. In fact, in the inference operating step the method for the analysis of radiographs receives as input analysis radiographic images, even if never acquired previously, and detects the elements present in them, as better defined below, regarding morphological deviations from typical patterns, providing the coordinates of anatomical points of interest.

Preferably, said two operating modes are alternated over time, so as to have models that are always updated, to reduce the detection errors that the analysis method could in any case commit.

The various steps of the operating method of the system for analysing lateral-lateral teleradiographs of the skull, divided into said two specified operating modes, are discussed below.

### A. Learning (Training)

Making reference to FIG. 1, it illustrates the main steps and sub-steps of the method for the analysis of lateral-lateral teleradiographs of the skull according to the present invention, when the learning operating step is carried out.

The learning operating step, indicated by the reference number 1, acquires as input (step 11) the learning radiographic images and the relative annotations, structured in the terms indicated above and, for every model to be learned, carries out one or more learning procedures.

Again in reference to FIG. 1, it is possible to distinguish between two main learning procedures or steps:
the radiograph cutout model learning procedure 12, wherein the cutout model is capable of detecting and cutting out the area of interest of the lateral-lateral teleradiograph of the skull for the purposes of cephalometric analysis;
the general model learning procedure 13, wherein the general model is capable of detecting the 60 cephalometric points listed, by way of example, in the table shown below; and
the refinement model learning procedure 14, which is applied for one or more refinement models. In particular, every radiograph is divided into areas and each area comprises a group of anatomical points of interest. The image analysis method has a refinement model for each group of anatomical points of interest thus created, which comprises anatomical points of interest that are close to one another. Each refinement model refines the output obtained from the general model, thus seeking to reduce error. In one embodiment, by way of example, there are 10 refinement models; however, the number of refinement models can be different and undergo variations on the basis, for example, of computational performance needs.

For each procedure carried out, various pre-processing and data augmentation operations are carried out, after which the actual learning (or so-called training) of the model takes place.

In an experimental setup for the learning procedures, for learning both the general model and the refinement models, use was made of 488 lateral-lateral teleradiographs of the skull, produced by different X-ray machines on various patients of different ages.

The annotation process was performed manually by a team of two expert orthodontists and consisted in marking the anatomical points of interest on the lateral-lateral teleradiographs of the skull by means of a computerised support.

### Learning of the Radiograph Cutout Model

As mentioned above, this preliminary learning procedure acquires, as input, the learning radiographic images and the annotations, as indicated in step 11, and returns a radiograph cutout model capable of detecting, starting from a lateral-lateral teleradiograph of the skull, the area that is relevant for cephalometric analysis.

Again in reference to FIG. 1, in the present radiograph cutout model learning procedure 12, the following sub-steps are carried out. The first sub-step is data augmentation 121, which in turn comprises the following sub-steps:
random rotation 1211, which is the first data augmentation sub-step for learning of the general model, wherein each image and the relative annotations can undergo a rotation with a probability, in the present embodiment, of 0.7. Naturally, in other embodiments there may be other probability values. If an annotated image is selected for rotation, from 1 to 10 generated images are obtained by rotating the original image by a rotation angle α, where α∈[−30°, +30°]. In further embodiments, the rotation angle α can also take on other values, for example, α∈[−45°, +45° ]. This operation makes it possible to take into consideration the fact that the learning radiographic images that will be subsequently acquired may have inclinations or random rotations when they are acquired;
random horizontal flip 1212, wherein the annotated images are randomly flipped horizontally with a probability of 0.5. In this case as well, in other embodiments the probability coefficient can be modified;
random contrast adjustment 1213, wherein the contrast of the images is adjusted based on a random factor with a value comprised between [0.7, 1.3]. In other embodiments, the random contrast adjustment factor of the present sub-step can be different;
random brightness adjustment 1214, wherein the brightness of images is adjusted based on a random factor comprised between [−0.2, 0.2]. In other embodiments, this random factor can be different;

Subsequently, after the data augmentation step 121, a resizing sub-step 122 is carried out wherein, in order to enable the execution of the learning algorithms, it is necessary to resize the images so that all parameters of the models can be contained in the memory. In some embodiments, the images are resized to 256×256.

Finally, a radiograph cutout model learning sub-step is carried out wherein the radiograph cutout model learning algorithms are executed. In this context, the learning consists in suitably setting the parameters of the deep learning model in order to minimize the cost functions.

In a preferred embodiment of the present invention, this model was built using an architecture of the Single Shot Detector (SSD) type (Liu, Reed, Fu, & Berg, 2016).

### Learning of the General Model

As mentioned above, this preliminary learning procedure acquires as input the learning radiographic images and annotations, as indicated in step 11, and returns a general model capable of detecting one or more anatomical points of interest, providing the coordinates relative to a reference system. In particular, in one embodiment, there are 60 anatomical points of interest, and they are shown in the following table.

Table of points of interest.

| # | Anatomical point |
|---|---|
| 1 | Frontal - external cortical of frontal |
| | (FRO) |
| 2 | Nasion (NA) |
| 3 | Anterior point of the nasal bone |
| | (NA1) |
| 4 | Superior orbital (ORBS) |
| 5 | Inferior orbital (ORB) |
| 6 | Anterior clinoidal apophysis (CLA) |
| 7 | Depression (SE) |
| 8 | Basion (BA) |
| 9 | Opisthion (OP) |
| 10 | Porion (PO) |
| 11 | Pterygoid (PT) |
| 12 | Inferior pterygoid (PTINF) |
| 13 | Posterior nasal spine (SNP) |
| 14 | Transverse incisive-canine suture |
| | (SNA1) |
| 15 | Anterior nasal spine (SNA) |
| 16 | Point A (A) |
| 17 | Apex of superior incisor root (RS) |
| 18 | Superior incisal (INS) |
| 19 | Inferior incisal (INI) |
| 20 | Apex of inferior incisor root (RI) |
| 21 | First superior premolar cusp |
| | (PREMS) |
| 22 | First inferior premolar cusp |
| | (PREMI) |
| 23 | Mesial cusp of first superior molar |
| | (MOLSM) |
| 24 | Distal cusp of first inferior molar |
| | (MOLSD) |
| 25 | Mesial cusp of first inferior molar |
| | (MOLICM) |
| 26 | Distal cusp of first inferior molar |
| | (MOLICD) |
| 27 | Point B (B) |
| 28 | Pogonion (PGO) |
| 29 | Suprapogonion (PM) |
| 30 | Gnathion (GNA) |
| 31 | Menton (MEN) |
| 32 | Posterior point of the posterior edge |
| | of the symphysis (SINP) |
| 33 | Inferior point of the anterior border of |
| | the mandibular ramus (R0) |
| 34 | Most inward point of the anterior |
| | border of the mandibular ramus (R1) |
| 35 | Coronoid process (COR) |
| 36 | Lowest, most median point of the |
| | sigmoid notch (R3) |
| 37 | Most anterior point of the mandibular |
| | condyle (CONMES) |
| 38 | Highest point of the mandibular |
| | condyle (CONCR) |
| 39 | Condilion (CONDIS) |
| 40 | Articular (ART) |
| 41 | Maximum concavity of the |
| | mandibular ramus (RAMOMAN) |
| 42 | Most posterior point of posteroinferior |
| | edge of the mandibular ramus |
| | (PREGO) |
| 43 | Gonion (GO) |
| 44 | Pregoniac notch (CORPOMAN) |
| 45 | Glabella (GLA) |
| 46 | Cutaneous nasion (NAS) |
| 47 | Nasal dorsum (DN) |
| 48 | Pronasale (PN) |
| 49 | Columella (COLUM) |
| 50 | Subnasale (SN) |
| 51 | A cutaneous (ACUT) |
| 52 | Superior labial point (UL) |
| 53 | Superior stomion (STMS) |
| 54 | Point of contact between upper lip |
| | and outer surface of superior incisor |
| | (ULINS) |
| 55 | Inferior stomion (STMI) |
| 56 | Lower lip point (LL) |
| 57 | Inferior sublabial point (SL) |
| 58 | Cutaneous pogonion (PGC) |
| 59 | Cutaneous menton (MEC) |
| 60 | Innermost point between submental |
| | area and neck (CP) |

Naturally, the number and type of points of interest can be different according to the preferred embodiment and the system's processing capability. In particular, the points to be detected, also as updated with the scientific literature, could change.

Again in reference to FIG. 1, the following sub-steps are carried out in the present general model learning procedure 13. The first sub-step is data augmentation 131, which in turn comprises the following sub-steps:
random rotation 1311, which is the first data augmentation sub-step for learning of the general model, wherein each image and the relative annotations can undergo a rotation with a probability, in the present embodiment, of 0.7. Naturally, in other embodiments one can have other probability values. If an annotated image is selected for rotation, 1 to 10 images are generated, obtained by rotating the original image by a rotation angle α, where α∈[−30°, +30°]. In further embodiments, the rotation angle α can also take on other values, for example, α∈[−45°, +45° ]. This operation makes it possible to take into consideration the fact that the learning radiographic images that will be subsequently acquired may have inclinations or random rotations when they are acquired,
random horizontal flip 1312, wherein the annotated images are randomly flipped horizontally with a probability of 0.5. In this case as well, in other embodiments the probability coefficient can be modified in other embodiments;
random contrast adjustment 1313, wherein the contrast of the images is adjusted based on a random factor with a value comprised between [0.7, 1.3]. In other embodiments, the random contrast adjustment factor of the present sub-step can be different;
random brightness adjustment 1314, wherein the brightness of images is adjusted based on a random factor comprised between [−0.2, 0.2]. In other embodiments, this random factor can be different;
random resizing and cutting out 1315, wherein the image is resized with a random scale factor comprised between [0.6, 1.3] and cut out (if part of the cutout does not fall within the image, it is filled with zeroes). In other embodiments, this random factor can be different.

Subsequently, after the data augmentation step 131, a resizing sub-step 132 is carried out wherein, in order to enable the execution of the learning algorithms, it is necessary to resize the images so that all parameters of the models can be contained in the memory. In some embodiments, the images are resized to 256×256.

Finally, a general model learning sub-step 133 is carried out wherein the general model learning algorithms are executed. In this context, the learning consists in suitably setting the parameters of the general deep learning model in order to minimize the cost functions.

In a preferred embodiment of the present invention, this model was built using an architecture of the CenterNet type (Zhou, Wang, & Krshenbuhl, 2019) based on Hourglass-104 (Law & Deng, 2018).

### Learning of the Refinement Models

As mentioned above, the general model, in one embodiment thereof, reduces the images to a size of 256×256 so that all the parameters can be contained in the memory. The image resolution and thus also the precision of the general model are reduced. The purpose of the refinement models is to improve the precision of the points of interest found by the general model, thereby reducing the errors due to the low resolution.

This refinement model learning procedure is composed of two essential steps. A first common step for all the models, which is thus carried out only once, and a model-specific step which is carried out several times, once for each refinement model to be learned.

### Common Step

The common step for all the refinement models comprises a data augmentation step 141, followed by an inference sub-step 142, wherein the general model obtained from the general model learning procedure 13 is exploited, and finally a cutting out sub-step 143, used to create the datasets necessary for the learning of the refinement models.

In particular, said data augmentation sub-step 141 comprises the following sub-steps:
random rotation 1411, for learning of the refinement models, wherein each image and the relative annotations can undergo a rotation with a probability of 0.7, wherein this value can be modified for other embodiments. If an annotated image is selected for rotation, from 1 to 10 generated images are obtained by rotating the original image by a rotation angle α, where α∈[−30°, +30° ]. In this case as well, in other embodiments different rotation angles α can be envisaged;
random horizontal flip 1412, wherein the annotated images are randomly flipped horizontally with a probability of 0.5, which can be varied in other embodiments;
random contrast adjustment 1413, wherein the contrast of the images is adjusted based on a random factor with a value comprised between [0.7, 1.3]. In this case as well, the range can be varied in other embodiments; and
random brightness adjustment 1414, wherein the brightness of images is adjusted based on a random factor comprised between [−0.2, 0.2]. This range can be varied in other embodiments.

As mentioned, an inference step is subsequently carried out by means of the general model learning procedure 13, which is indicated here as sub-step 142. In this case, all the learning radiographic images are provided as input to the general model learned in the general model learning procedure 13 and the 60 anatomical points listed in the above table are detected.

Subsequently, a step 143 of cutting out the learning radiographic image R, the object of processing, is performed wherein the points detected in the previous sub-step are grouped into N groups and, for each group, a cutout of the learning radiographic image R is generated, containing the points belonging to the group starting from the original learning radiographic image R. In one embodiment, N is equal to 10. The output of this sub-step is a plurality of N datasets, where N is the number of refinement models to be learned, one for every group of points.

In other words, the whole radiographic images are passed on to the general model to obtain the 60 (in the present embodiment) anatomical points of interest. Only after this step is the original image cut.

Given the i-th dataset, one example is composed of a pair <Rᵢ,e>, where Rᵢ is the cutout of the original learning radiographic image containing the points detected by the general model and e is the error vector in the form:

$$e = [ {d}_{{p}_{0} x} , {d}_{{p}_{0} y} , ⋯ , {d}_{{p}_{j} x} , {d}_{{p}_{j} y} , ⋯ , {d}_{{p}_{K} x} , {d}_{{p}_{K} y} ]$$

wherein K is the number of anatomical points refined by the i-th refinement model, dₚ_{ⱼ}ₓ is the difference between the real x coordinate of the point pⱼ and the one predicted by the general model and, similarly, dₚ_{ⱼ}_{y} is the difference for the y coordinate.

The learning of the refinement models has the aim of defining models that are able to approximate e starting from the image cutout Rᵢ.

### Specific Step

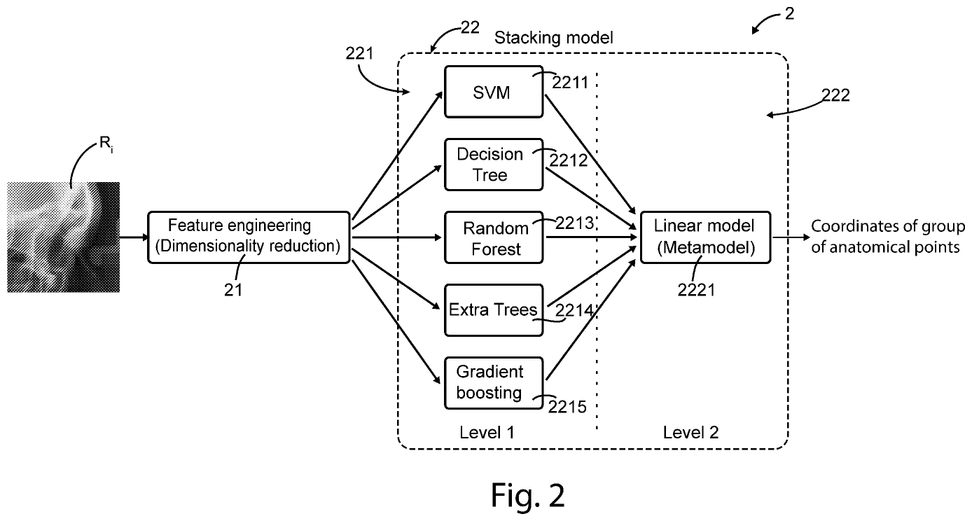

After the various cutouts have been obtained on the basis of the groups of points, i.e. the cutouts of the learning radiographic image R, for every refinement model 151 . . . 15N to be learned, the following sub-steps are carried out:
resizing 1511 . . . 15N1, wherein the images are resized in order to be able to be processed by the automatic learning algorithms. In some embodiments, the images are resized to 256×256;
learning of models for feature engineering and refinement model 2; a refinement model with a structure similar to the one shown in FIG. 2, and better described below, is trained in this sub-step 2. In particular, for feature engineering, it is possible to carry out feature extraction methods (such as models based on convolutional neural networks or computer vision algorithms for the extraction of Haar-like features or histogram of oriented gradients (HOG)), and dimensionality reduction methods, such as, for example, Partial Least Squares (PLS) regression or Principal Component Analysis (PCA). In some embodiments, feature engineering takes place in two steps: in the first step, the features are extracted from the image using the histogram of oriented gradients (HOG) algorithm, whereas in the second, in order to reduce the dimensionality of the examples (dimensionality reduction), a Partial Least Squares (PLS) regression model is trained or a feature engineering procedure 21 is carried out, after which a set of regression models (ensemble model) 22 is trained with the two-level stacking technique, comprising a first 221 and a second 222 level. In one embodiment, the following are used as first-level models 221: support vector machine (SVM) 2211, decision trees 2212, random forest 2213, extra tree 2214 and gradient boosting 2215, whereas a linear regression model with coefficient regularization 2221 is used as the final second-level model (also called metamodel). In different embodiments, these models could vary, be reduced or include further models. As may be observed, the coordinates of the group of anatomical points or points of interest are obtained as output.

### B. Inference

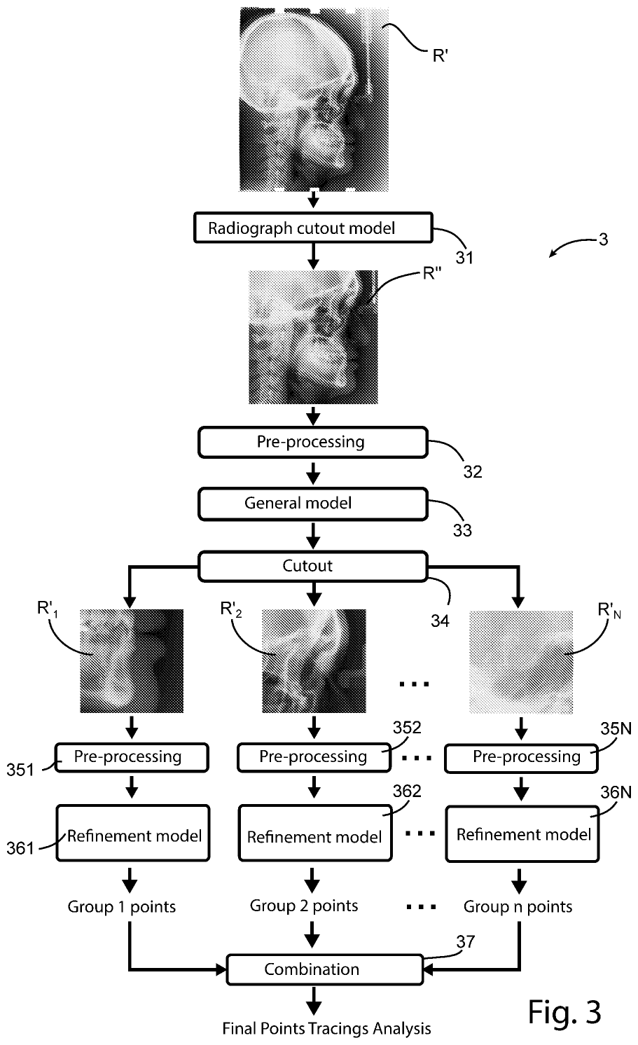

The inference operating step, shown and illustrated in FIG. 3 and indicated by the reference number 3, receives as input a whole, completely new lateral-lateral analysis radiographic image R′ of the skull and returns as output the anatomical points of interest detected, that is, the coordinates thereof relative to a reference system. In addition, based on the points detected, in a post-processing step, the method of analysis according to the invention can define cephalometric tracings and perform cephalometric analyses.

In particular, the main sub-steps of the inference operating procedure 3 are specified below.

Initially, an inference step 31 is carried out for the radiograph cutout model, wherein the original lateral-lateral teleradiograph of the skull R′ is provided as input to the radiograph cutout model, which identifies the area of interest for cephalometric analysis and cuts out the radiograph so as to obtain the image R″.

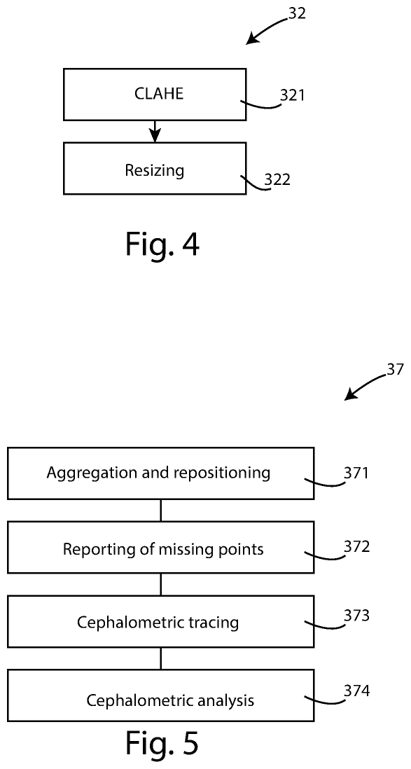

Subsequently, a pre-processing step 32 for the general model is carried out, which comprises (see FIG. 4) contrast limited adaptive histogram equalization (CLAHE) 321, wherein the image is modified in contrast. This operation is optional. Subsequently, there is a resizing step 322, wherein the new radiographic image R″, in some embodiments, is resized to 256×256 in order be processed by the model obtained in the learning step.

Subsequently, in step 33, an inference step is carried out based on the general model learned in the general model learning procedure 13, wherein the pre-processed analysis radiographic image R″ is input to a general deep learning model obtained from the first learning procedure, which, in an embodiment thereof, returns the geometric coordinates of the 60 points listed in the table above.

Subsequently, a cutting out step 34 is performed; the points obtained in the previous inference step are organized into N groups and, for every group of points detected, a cutout R′₁, R′₂, . . . , R′_{N} containing the group of points detected is generated from the original analysis radiograph image R′. The width and height of the cutout generated are preferably at least 256 pixels. The grouping of points or image cutouts is similar to those of the cutting out sub-step 143 described above.

For every refinement model, with reference to FIGS. 2 and 3, the following sub-steps are carried out:
pre-processing for refinement model 351 . . . 35N, wherein the image is resized and the algorithms and models for feature engineering obtained in the learning step are applied to the cutout (see FIG. 2);
inference by means of the refinement model 351 . . . 35N; the output of the pre-processing step is given as input to the first-level models and the outputs of the first-level models are passed on as input to the final second-level model.

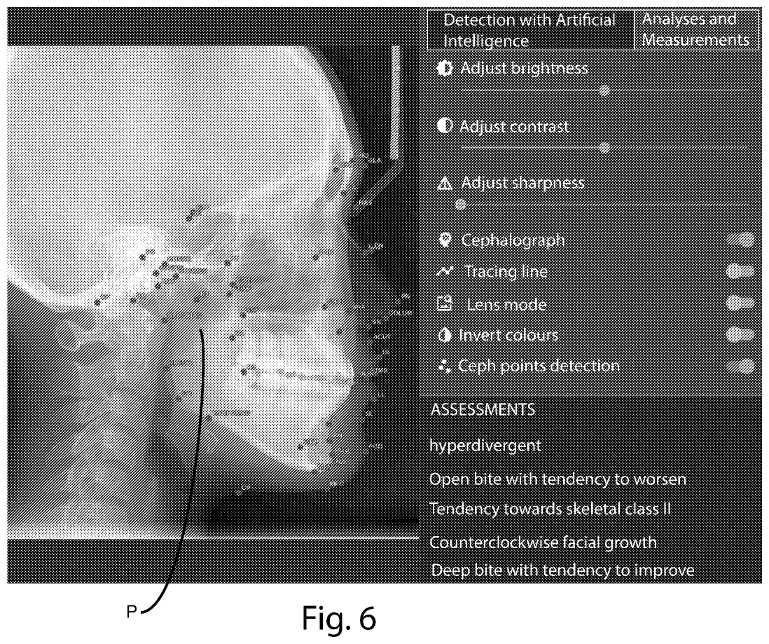

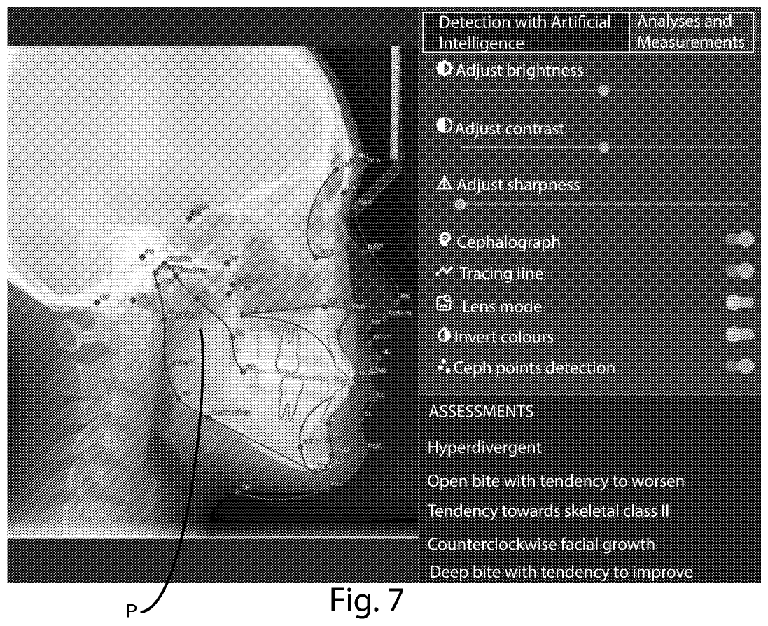

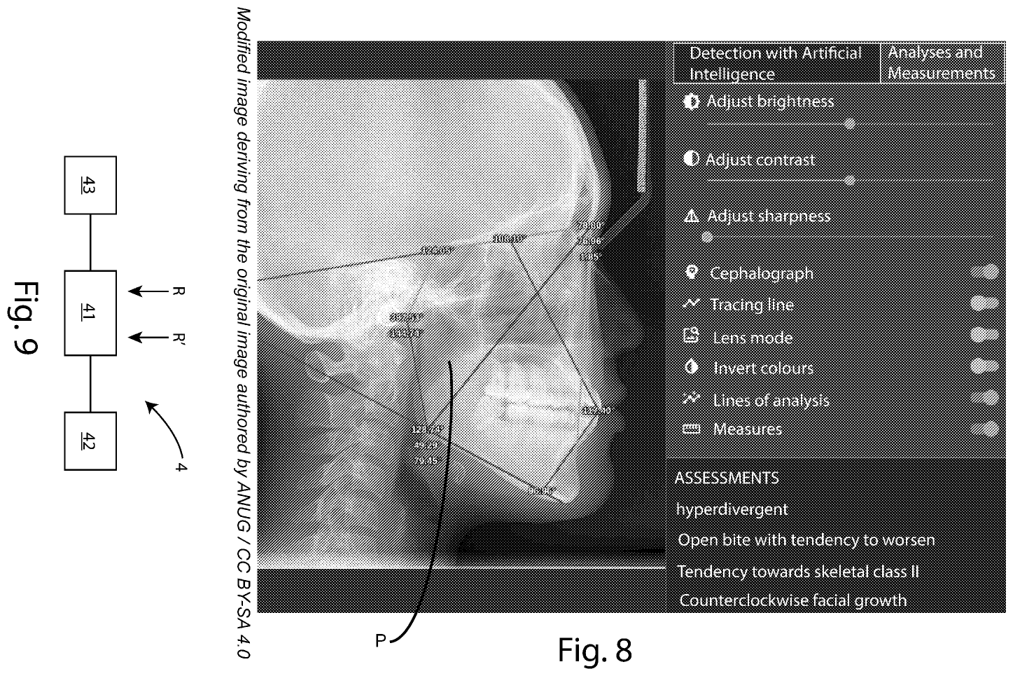

From each inference sub-step, by means of the refinement model 361 . . . 36N (which represents the predicted error of the general model), one obtains the points of each group 1 . . . N, relative to each cutout R′₁, R′₂, . . . , R′_{N} of the analysis radiographic image R′. These groups of points of the cutouts R′₁, R′₂, . . . , R′_{N} are combined in a post-processing step with the outputs of the general model (combining step 37), in order to have final geometric coordinates of the points relative to the new, original radiographic image R′. In particular, the post-processing for carrying out the combining step comprises the following sub-steps (see FIG. 5):
aggregation and repositioning 371 of the anatomical points, wherein the annotations returned by the refinement models are aggregated together with those of the original model, in such a way that the geometric coordinates of the anatomical points detected are relative to the original radiographic image R′. A visual example of the anatomical points detected by the execution of the models is shown in FIG. 6;
reporting of missing points 372, wherein it is reported whether there are points that have not been detected;
cephalometric tracing 373, wherein, the tracing lines are defined based on the points detected. A visual example of the definition of the cephalometric tracing (following the detection of the anatomical points) is shown in FIG. 7;
cephalometric analysis 374, wherein, based on the points detected, one or more cephalometric analyses among the ones known in the literature are performed. A visual example of the Jarabak cephalometric analysis (following the detection of the anatomical points) is shown in FIG. 8.

In particular, as may be observed in FIGS. 6-8, the anatomical points P, based on which the cephalometric analyses are performed, are highlighted.

In particular, FIGS. 6-8 show an interface viewable on a computer monitor, by means of which the doctor or operator can perform analyses of the appropriate diagnoses.

Finally, making reference to FIG. 9, one observes a general diagram of a system for the analysis of lateral-lateral teleradiographs of the skull, indicated by the reference number 4, comprising a logical control unit 41, which receives as input the learning radiographic images R and the analysis radiographic images R′, and comprising processing means, such as a processor and the like, configured to carry out the above-described method for the analysis of lateral-lateral teleradiographs of the skull.

Furthermore, the system 4 comprises interaction means 42, which can include a keyboard, a mouse or a touchscreen, and display means 43, typically a monitor or the like, to enable the doctor to examine the processed images and read the coordinates of the anatomical points of interest, in order possibly to derive appropriate diagnoses.

By means of the display means 43 it is possible to display the anatomical points of interest after the processing has been performed and to examine the geometric arrangement thereof.

### Advantages

One advantage of the present invention is that of providing a support for doctors, radiologists and dentists in particular, which makes it possible to detect and locate anatomical points of the skull which are useful for cephalometric analysis.

A further advantage of the present invention is that of enabling the practitioner to carry out correct diagnoses and therapies, thus enabling accurate treatments.

Another advantage of the present invention is that of enabling an automatic analysis of the analysis radiographic images such as to enable the obtainment of data for in-depth epidemiologic studies and analyses of the success of dental treatments.

### REFERENCES

Law, H., & Deng, J. (2018). Cornernet: Detecting objects as paired keypoints. Proceedings of the European conference on computer vision (ECCV), (p. 734-750).
Liu, W. a., Reed, S., Fu, C.-Y., & Berg, A. C. (2016). SSD: Single Shot Multibox Detector. Computer Vision—ECCV 2016: 14th European Conference, Amsterdam, The Netherlands, October 11-14, 2016, Proceedings, Part the 14 (p. 21-37). Springer.
Zhou, X., Wang, D., & Krähenbühl, P. (2019). Objects as points. arXiv preprint arXiv:1904.07850.

The present invention has been described by way of non-limiting illustration according to the preferred embodiments thereof, but it is to be understood that variations and/or modifications may be introduced by the person skilled in the art without for this reason going outside the relevant scope of protection as defined by the appended claims.

The invention claimed is:

1. A method for a computer-implemented geometric analysis of digital radiographic images (R) using a radiographic system (4), wherein said radiographic system (4) comprises:
a display unit (43); and
processing means (41), connected to said display unit (43),
said method comprising the steps of:
performing with said processing means (41) a learning step (1) comprising the following sub-steps:
receiving (11) a plurality of digital learning radiographic images, each accompanied by annotations, wherein an annotation comprises a label identifying an anatomical point of interest of each learning radiographic image (R), and geometric coordinates of the anatomical point of interest in a plane of a learning radiographic image (R);
executing (13), with said processing means (41) for each learning radiographic image (R), a general model learning procedure for learning a general model for detecting one or more points of interest from the learning radiographic image (R); and
performing a refinement model learning procedure (14), comprising the sub-steps of:
cutting (143) the learning radiographic image into a plurality of image cutouts (R₁, R₂, . . . , R_{N}), each comprising a respective group of anatomical points of interest; and
training (151 . . . 15N) a refinement model (2) for each image cutout (R₁, R₂, . . . , R_{N}); and
carrying out an inference step (3) using said processing means (41) on a digital analysis radiographic image (R′), comprising the following sub-steps:
performing (33) on said analysis radiographic image (R′) an inference step based on said general model learned in said general model learning procedure, so as to obtain geometric coordinates of a plurality of anatomical points of interest;
cutting (34) the analysis radiographic image (R′) into a plurality of image cutouts (R′₁, R′₂, . . . , R′_{N}) as in the cutting step (143) of the learning radiographic image, wherein each image cutout (R′₁, R′₂, . . . , R′_{N}) of the analysis radiographic image (R′) comprises a respective group of anatomical points of interest; and
performing (361 . . . 36N) on each cutout of the analysis radiographic image (R′) an inference through said refinement model obtained in said training step (151 . . . 15N) of said refinement model learning procedure (14); and
combining (37) the anatomical points of interest of each image cutout (R′₁, R′₂, . . . , R′_{N}) of the analysis radiographic image (R′) so as to obtain final geometric coordinates of points relative to the original analysis radiographic image (R′); and
displaying said final geometric coordinates of the points relative to the original analysis radiographic image (R′) with said display unit (43).

2. The method according to claim 1, wherein said learning step (1) comprises a sub-step of performing (12), with said processing means (41), for each learning radiographic image (R), a procedure for learning a radiograph cutout model for cutting out a part of a lateral-lateral teleradiograph of a skull that is relevant for cephalometric analysis.

3. The method according to claim 2, wherein carrying out said inference step (3) comprises a sub-step of performing (31), on said analysis radiographic image (R′), the inference step based on said radiograph cutout model learned in said radiograph cutout model learning procedure (12), so as to obtain a cutout of the part of the lateral-lateral teleradiograph of the skull relevant for the cephalometric analysis (R″).

4. The method according to claim 3, further comprising a step of performing (31), on said analysis radiographic image (R′), the inference step based on said radiograph cutout model, which is carried out before said step of performing (33) on said analysis radiographic image (R″) an inference step based on said general model learned in said general model learning procedure (13).

5. The method according to claim 1, wherein said general model learning procedure (13) comprises a first data augmentation step (131) comprising the following sub-steps:
random rotation (1311) of the radiographic image (R) by a predefined range of angles with predefined probability;
random horizontal flip (1312), wherein the acquired radiographic images (R) with the annotations are randomly flipped horizontally with a predefined probability;
random contrast adjustment (1313), wherein image contrast is adjusted based on a predefined random factor;
random brightness adjustment (1314), wherein a brightness of images is adjusted based on a predefined random factor; and
random resizing and cutting out (1315, 1316), wherein the radiographic image (R) is resized with a random scale factor and cut out.

6. The method according to claim 5, wherein said general model learning procedure (13) comprises a resizing sub-step (132).

7. The method according to claim 1, wherein said refinement model learning procedure (14) comprises the sub-steps of:
performing a second data augmentation step (141); and
executing said general model (13) as obtained from said general model learning procedure.

8. The method according to claim 7, wherein said second data augmentation step (141) of said refinement model learning procedure (14) comprises the following sub-steps:
random rotation (1411), wherein each radiographic image (R) and related annotations are rotated by a predefined range of angles and/or with a predefined probability, generating a plurality of rotated images;
horizontal flip (1412) of the radiographic images (R) randomly annotated with a predefined probability;
adjusting contrast (1413) of said radiographic images (R) based on a predefined random factor; and
adjusting the contrast (1414) of said radiographic images based on a predefined random factor.

9. The method according claim 1, wherein said step of training (151 . . . 15N) a refinement model for each image cutout (/?i,/?₂>->RN) comprises the following sub-steps:
resizing (1511 . . . 15N1) each cutout of said radiographic image (/?_{£}), and carrying out a feature engineering and refinement model learning procedure (2); and/or
carrying out a dimensionality reduction model learning procedure, and carrying out the refinement model learning.

10. The method according to claim 9, wherein said step of carrying out a feature engineering and refinement model learning procedure (2) is based on computer vision algorithms, or on deep learning procedures.

11. The method according to claim 9, wherein said step of carrying out a dimensionality reduction model learning procedure comprises Principal Component Analysis (PCA) or Partial Least Squares regression (PLS).

12. The method according to claim 11, wherein said step of carrying out a feature engineering and refinement model learning procedure (2) comprises:
a feature engineering model or procedure (21); and
a set of regression models (22) with a two-level stacking technique, comprising,
a first level (221), comprising one or more models, and
a second level (222) comprising a metamodel (2221) and
wherein at an output of said refinement model learning procedure (2), coordinates of a group of anatomical points or points of interest of each cutout of said radiographic image (Rᵢ) are obtained.

13. The method according to claim 12, wherein said one or more models of said set of regression models (22) comprise at least one of the following models: support vector machine (2211); and/or decision trees (2212); random forest (2213); extra tree (2214); or gradient boosting (2215).

14. The method according to claim 1, wherein a step (32) of pre-processing said analysis radiographic image (R′) comprises the following sub-steps:
performing an adaptive equalization of a contrast-limited histogram (321), wherein the image is modified in contrast; and
resizing (322) the analysis radiographic image (R′).

15. The method according to claim 1, wherein said combining step (37) of said inference step (3) comprises the steps of:
aggregating and repositioning (371) the anatomical points of interest, wherein the annotations returned by the refinement models are aggregated together with the annotations of the original model, in such a way that geometric coordinates of the anatomical points detected are relative to the original analysis radiographic image (R′);
reporting (372) missing anatomical points of interest, wherein the reporting comprises reporting whether there are points that have not been detected;
carrying out a cephalometric tracing (373), wherein, based on the detected points, tracing lines are defined; and
performing a cephalometric analysis (374), wherein, based on the detected points, one or more cephalometric analyses among cephalometric analyses known in scientific literature are performed.

16. A system for analyzing digital radiographic images, comprising
a display unit (43); and
processing means (41), connected to said display unit (43), configured to carry out the method according to claim 1.

17. A computer program comprising instructions which, when the computer program is executed by a computer, cause the computer to execute the steps of the method according to claim 1.

18. A computer readable storage medium comprising instructions which, when executed by a computer, cause the computer to execute the steps of the method according to claim 1.
